(19) United States
(12) Patent Application Publication
(10) Pub. No.: US 2023/0233776 A1
(43) Pub. Date: Jul. 27, 2023

(54) INJECTION GUIDANCE TEMPLATE

(71) Applicant: Silvia Viktoria Rabiniak, Richmond (CA)

(72) Inventor: Silvia Viktoria Rabiniak, Richmond (CA)

(21) Appl. No.: 17/586,745

(22) Filed: Jan. 27, 2022

(51) Int. Cl. A61M 5/42

(52) U.S. Cl. CPC A61M 5/427

(57) ABSTRACT

An injection guidance template operable to provide proper land-marking of a syringe needle for execution of an intramuscular deltoid injection. The present invention includes a body wherein the body is planar and manufactured from a material that is impenetrable with a syringe needle. The body includes a preferred shape having three contiguous sections providing a bowtie like shape. The center section of the body includes a centrally located aperture. The aperture is present so as to provide a location for insertion of a needle into a patient having the present invention on their shoulder. The body includes a first surface and a second surface wherein the second surface is placed adjacent the skin of a patient and can further include an adhesive to releasably secure thereto. The first surface of the body can further include decorative indicia.

### FIELD OF THE INVENTION

The present invention relates generally to healthcare administration, more specifically but not by way of limitation, a template configured to be releasably secured and/or positioned on a shoulder of a patient receiving an injection such as but not limited to vaccine wherein the present invention guides the practitioner to proper placement of the syringe needle.

### BACKGROUND

Millions of injections are administered to patients everyday. Whether its vaccines or other medicinal therapies, it is common practice to utilize a syringe to perform administration thereof. When not executed properly, the patient can be at risk for shoulder injury during administration of the injection. Shoulder injury occurs in a significant portion of individuals that receive a vaccine or other type of injection. Shoulder injury related to injections are a preventable occurrence that is caused by the injection of a vaccine or other medicine into the shoulder capsule rather than the deltoid muscle. When this occurs it results in inflammation of the shoulder structure and causes patients to experience pain and decreased range of motion. Shoulder injuries related to vaccine administration and other injections go largely un-reported and are most always preventable. These injuries are caused by incorrect technique or land-marking for intramuscular deltoid injections.

Shoulder injuries occur when an intramuscular deltoid injection is administered onto the shoulder joint. This results in an inflammation that causes damage to the musculoskeletal structures includes the bursae, tendons and ligaments. The main symptoms include persistent shoulder pain and a limited range of motion. Additionally, injections that occur below the deltoid muscle can hit the radial nerve and injections that are to far to the side of the deltoid muscle can hit the axillary nerve. When a nerve is hit the patient will feel an immediate burning pain and this can results in neuropathy or other issues. Proper land-marking of the injection site includes determination of the injection zone of the deltoid muscle to include upper and lower borders of a safe injection area. This technique has been developed over the years in order to avoid injury to the patient. The upper border is typically founds two to three finger widths from the acromion which insures injection below the shoulder capsule. The lower border is often marked by the armpit to ensure the injection is not administered below the deltoid. Despite existing guidelines as discussed herein above, the prevalence of shoulder injuries due to improper land-marking of the injection site is still quite high.

Accordingly, there is a need for a guidance template that provides a practitioner proper land-marking of an injection site for a deltoid intramuscular injection in order to avoid a potential shoulder injury.

### SUMMARY OF THE INVENTION

It is the object of the present invention to provide a guidance template for an injection site to facilitate proper needle location for a deltoid intramuscular injection wherein the present invention includes a body that is planar in manner.

Another object of the present invention is to provide a template to assist in the proper execution of an intramuscular deltoid injections wherein the body includes a first section, a second section and a third section.

A further object of the present invention is to provide a guidance template for an injection site to facilitate proper needle location for a deltoid intramuscular injection wherein the first section, second section and third section of the body are contiguously formed.

Still another object of the present invention is to provide a template to assist in the proper execution of an intramuscular deltoid injection wherein the second section of the body is intermediate the first section and third section.

An additional object of the present invention is to provide a guidance template for an injection site to facilitate proper needle location for a deltoid intramuscular injection wherein the body include a first surface and a second surface wherein the second surface is placed adjacent a patient shoulder during administration of an injection.

Yet a further object of the present invention is to provide a template to assist in the proper execution of an intramuscular deltoid injection wherein the second surface of the body can include a pressure sensitive adhesive thereon.

Another object of the present invention is to a provide a guidance template for an injection site to facilitate proper needle location for a deltoid intramuscular injection wherein the second section includes an aperture centrally located thereon.

An alternate object of the present invention is to provide a template to assist in the proper execution of an intramuscular deltoid injection wherein the body is manufactured from a material that is impenetrable by a needle.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a side perspective view of the present invention on an exemplary shoulder; and

FIG. 2 is a front view of the present invention secured to an exemplary patient shoulder.

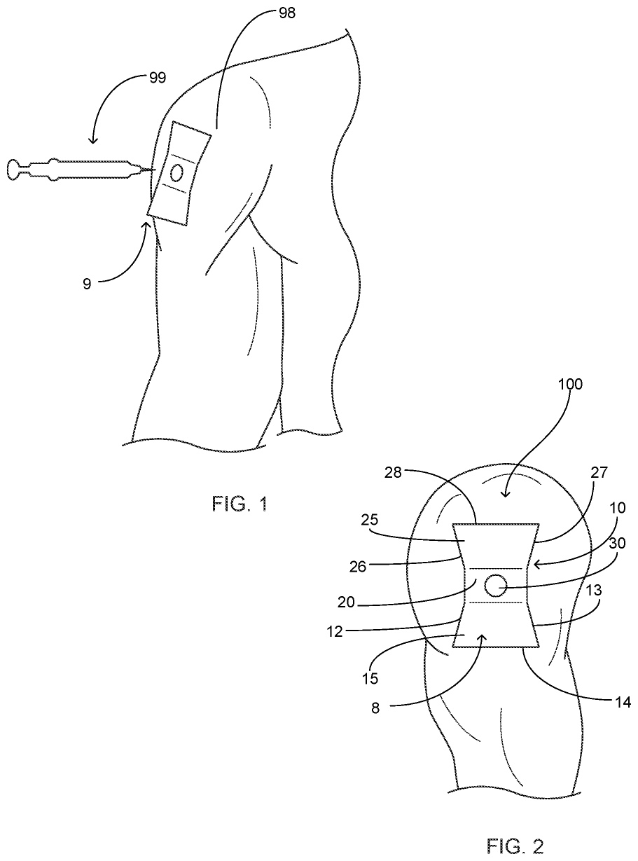

### DETAILED DESCRIPTION

Referring now to the drawings submitted herewith, wherein various elements depicted therein are not necessarily drawn to scale and wherein through the views and figures like elements are referenced with identical reference numerals, there is illustrated an injection guidance template 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Now referring in particular to the Figures submitted herewith, the injection guidance template 100 includes body 10 wherein the body 10 is manufactured from a material that is impenetrable to a syringe needle 99. It should be understood within the scope of the present invention that various suitable materials could be employed to construct the body 10 such as but not limited to plastic. In a preferred embodiment the body 10 is relatively thin having a thickness of three to five millimeters. Thicknesses of the body 10 can vary dependent upon the material utilized to manufacture the body 10.

The body 10 includes a first section 15, a second section 20 and a third section 25 all contiguously formed to create the shape of the body 10. It should be understood within the scope of the present invention that the preferred embodiment of the body 10 has a shape that is illustrated herein however, alterations thereof are contemplated within the scope of the present invention. By way of example but not limitation, the body 10 could be comprised of a single section having an alternate shape such as but not limited to rectangular. The second section 20 is intermediate the first section 15 and third section 25. The second section 20 has a width that is less than that of the widths of the first section 15 and third section 25. The second section 20 incudes a center aperture 30. It is the center aperture 30 that provides guidance to the user of the injection guidance template 100 for proper land-marking of the syringe needle 99. The location of the center aperture 30 subsequent placement of the body 10 on a shoulder 98 provides the correct location for execution of an intramuscular deltoid injection. While it is contemplated within the scope of the present invention that the center aperture 30 could be provided in alternate sizes, a center aperture 30 having a diameter of a half inch is a preferred size. It should be further understood within the scope of the present invention that the center aperture 30 could be formed in an alternate shape as opposed to annular.

The first section 15 includes opposing lateral edges 12,13 that extend from the second section 20 to the outer edge 14 of the first section 15. The opposing lateral edges 12,13 are angular in orientation to create the shape of the first section 15 so as to have a width proximate the outer edge 14 that is greater than the width proximate the second section 20. This preferred shape helps protect from accidental injections into undesired areas of the shoulder 98.

The third section 25 includes opposing lateral edges 26,27 that extend from the second section 20 to the outer edge 28 of the third section 25. The opposing lateral edges 26,27 are angular in orientation to create the shape of the third section 25 so as to have a width proximate the outer edge 28 that is greater than the width proximate the second section 20. This preferred shape helps protect from accidental injections into undesired areas of the shoulder 98.

The body 10 includes a first surface 8 and a second surface 9. The second surface 9 is placed adjacent the skin and it is contemplated within the scope of the present invention that the second surface 9 could have an adhesive thereon configured to temporarily hold the injection guidance template 100 in place. It should be understood within the scope of the present invention that the second surface 9 could be provided with or without an adhesive. In an embodiment of the injection guidance template 100 it is contemplated within the scope of the present invention that the first surface 8 could have indicia thereon such as but not limited to comic super heros or other indicia that would be considered child friendly and/or entertaining. It should be understood within the scope of the present invention that the body 10 could be provided in alternate sizes so as to accommodate various shoulder sizes of adult and children so as to ensure proper land-marking by the center aperture 30.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding description is, therefore, not intended to be limited to the specific forms set forth herein, but on the contrary, it is intended to cover such alternatives, modifications, and equivalents, as can be reasonably included within the spirit and scope of the invention.

What is claimed is:

1. An injection guidance template for ensuring proper injection location for an intramuscular deltoid injection wherein the injection guidance template comprises:
a body, said body having at least one section, said body being planar in manner, said body having a first surface and a second surface, said body being manufactured from a material impenetrable by a syringe needle, said body configured to provide coverage of at least a portion of a deltoid muscle subsequent said body being placed thereagainst; and
an aperture, said aperture being centrally located on said body, said aperture being of sufficient diameter so as to allow the syringe needle to pass therethrough.

2. The injection guidance template for ensuring proper injection location for an intramuscular deltoid injection as recited in claim 1, wherein said aperture is annular in shape.

3. The injection guidance template for ensuring proper injection location for an intramuscular deltoid injection as recited in claim 2, wherein said body includes a lower end and an upper end, said body having a width proximate said lower end and said upper end that is greater than a width at a center thereof.

4. The injection guidance template for ensuring proper injection location for an intramuscular deltoid injection as recited in claim 3, wherein said second surface further includes an adhesive disposed on a portion thereof.

5. The injection guidance template for ensuring proper injection location for an intramuscular deltoid injection as recited in claim 4, wherein said body is manufactured from plastic.

6. An injection guidance template configured to provide proper land-marking for an injection on a deltoid muscle for an intramuscular injection wherein the injection guidance template comprises:
a body, said body being planar in manner, said body having a first section, a second section and a third section, said second section being intermediate said first section and said third section, said first section, said second section and said third section being contiguously formed, said first section having opposing lateral edges, said opposing lateral edge being angular in orientation and extending outward from said second section, said third section of said body having opposing lateral edges, said opposing lateral edges of said third section of said body being angular with respect to said second section and extending outward therefrom;
a central aperture, said central aperture being located in said second section of said body, said central aperture operable to mark a location suitable for an injection, said central aperture being of suitable diameter to permit a syringe needle to pass therethrough.

7. The injection guidance template configured to provide proper land-marking for an injection on a deltoid muscle as recited in claim 6, wherein said body further includes a first surface and a second surface, said second surface being adjacent skin of a patient ensuing placement of the injection guidance template.

8. The injection guidance template configured to provide proper land-marking for an injection on a deltoid muscle as recited in claim 7, wherein said body has a width proximate an upper edge on said third section that is greater than a width of the second section.

9. The injection guidance template configured to provide proper land-marking for an injection on a deltoid muscle as recited in claim 8, wherein said first surface includes decorative indicia imprinted thereon.

10. The injection guidance template configured to provide proper land-marking for an injection on a deltoid muscle as recited in claim 9, wherein said body has a width proximate a lower edge of said first section that is greater than the width of the second section.
